(19) United States
(12) Patent Application Publication
(10) Pub. No.: US 2022/0305183 A1
(43) Pub. Date: Sep. 29, 2022

(54) VASCULAR ARTERIOVENOUS GRAFT

(71) Applicant: INNAVASC MEDICAL, INC., Durham, NC (US)

(72) Inventors: Shawn M. Gage, Raleigh, NC (US); Craig Nichols, Durham, NC (US); Joseph Knight, Durham, NC (US); Juan Chiossone, Raleigh, NC (US); Peter Lawson, Durham, NC (US); Samantha Busch, Indian Land, SC (US); Brian Walsh, Charlotte, NC (US)

(21) Appl. No.: 17/566,428

(22) Filed: Dec. 30, 2021

Related U.S. Application Data
(60) Provisional application No. 63/166,790, filed on Mar. 26, 2021.
(60) Provisional application No. 63/166,794, filed on Mar. 26, 2021.

(51) Int. Cl. A61M 1/36; A61M 39/04; A61M 39/02; A61M 39/00

(52) U.S. Cl. CPC A61M 1/3655; A61M 1/3661; A61M 39/04; A61M 39/0208; A61M 2039/0036; A61M 2205/0216

(57) ABSTRACT

A cannulation chamber is provided for use with an arteriovenous graft including a flexible conduit. The cannulation chamber comprises an elongated body defining an annular passageway having a longitudinal axis extending between a first end and a second end. The body receives and surrounds the conduit in the passageway. The body comprises a self-sealing material and a cannulation port that exposes the self-sealing material. A flexible resilient elongated back plate is embedded in the body of the cannulation chamber such that the back plate extends generally parallel with and may partially surround the passageway. The back plate is formed of a substantially rigid material such that when a needle is inserted through the cannulation port and the self-sealing material, the needle is inhibited or prevented from extending through the back plate.

### CROSS-REFERENCES TO RELATED APPLICATIONS

This application claims priority to U.S. Provisional Patent Application No. 63/166,790 and U.S. Provisional Application No. 63/166,794, both filed Mar. 26, 2021, the contents of both of which are incorporated by reference herein in their entirety.

### GOVERNMENT RIGHTS

This invention was made with government support under contract number NSF Award 1951020 awarded by the National Science Foundation The government has certain rights in the invention.

### BACKGROUND

Vascular grafts are described and, more particularly, arteriovenous grafts used for hemodialysis.

A common technique to provide vascular access for hemodialysis is to connect a prosthetic graft or shunt between an artery and a vein in, for example, an upper or lower extremity. Occasionally, patient complexity may also warrant access placement on the chest or abdominal wall. A conventional prosthetic arteriovenous graft (AVG) is often constructed of a polymeric material, such as expanded polytetrafluoroethylene (ePTFE) or polyetherurethane.

A significant mode of failure of an arteriovenous graft is related to a traumatic cannulation with the dialysis needle. This may occur as the needle traverses the anterior wall of the arteriovenous graft and then continues through the posterior wall or a sidewall of the graft. This type of trauma causes a defect in the posterior or side wall of the graft and often results in hematoma formation which can ultimately lead to graft thrombosis (i.e., the formation of a blood clot inside the graft, obstructing the flow of blood therethrough) by external compression of the graft and ultimately graft failure.

The aforementioned cannulation related complications mentioned above can also be compounded when the vascular access is difficult to locate under the skin, which can be a common issue associated with hemodialysis vascular access. Difficult to locate vascular access leads to a significant amount of anxiety for both the cannulator and the patient because it is well understood by both dialysis technician/nurse and patient that a mis-cannulation event can lead to significant morbidity (e.g. hematoma, bleeding, pain, or swelling) as well as missed dialysis sessions.

Moreover, repeated punctures of the graft material, such as the ePTFE, promotes coring and degeneration of the graft material which often leads to rupture of the graft, pseudoaneurysm formation, and graft thrombosis. This degenerative process can be accelerated considerably if used in a home hemodialysis (HHD) setting because in order to achieve some of the most important benefits of HHD, treatment is typically performed 4-6 times per week, generally doubling the number of graft punctures performed per week when compared to conventional, in-center hemodialysis. Also, ePTFE grafts are generally not self-sealing when punctured and usually require implantation three, four or more weeks prior to initial puncture to allow for graft incorporation in which a layer of fibrotic tissue that attaches to the outside surface of the graft.

Some of these problems have been solved by incorporating rigid or semi-rigid structure into the AVG such that a needle cannot penetrate past the interior of the arteriovenous graft. For example, self-sealing vascular access grafts have been described in U.S. Pat. No. 5,192,310, and the problem of puncturing posterior or side walls of the grafts is contemplated in U.S. Pat. Nos. 6,261,257 and 9,585,998, the contents of all three of which are hereby incorporated by reference in their entirety. However, given the tight bends required for an arteriovenous graft to be deployed in the extremity of the subject, such upper or lower arm, the rigidity of the arteriovenous graft can kink in the area of the rigid structure or may simply not allow for the clinician to adequately bend the chamber during implantation. As a result, puncture-resistant chambers that are substantially straight or are not curved to the proper degree may not be used or will fail in certain applications. Moreover, the bending of grafts employing semi-rigid shields can weaken or kink the graft or disrupt flow characteristics for the blood flowing through the graft, even if the graft does not kink.

For the foregoing reasons, there is a need for an arteriovenous graft that is configured to be implanted in an upper or lower extremity of a patient and which is resistant to kinking, is easily identifiable, more durable to increased frequency of needle punctures, and prevents needle cannulation related complications. Ideally, the new graft will be self-sealing, resistant to inadvertent needle penetration and will also flex and bend without kinking and without otherwise effecting fluid flow through the graft.

### SUMMARY

A cannulation chamber is provided for use with an arteriovenous graft including a flexible conduit. The cannulation chamber comprises an elongated body having a first end and a second end and defining an annular passageway having a longitudinal axis extending between the first end and the second end. The body is adapted to receive and surround a least a portion of the conduit in the passageway. The body comprises a flexible, nonporous elastomeric self-sealing material, and a cannulation port that exposes the self-sealing material. A flexible resilient elongated back plate having a first end and a second end is embedded in the body of the cannulation chamber. The first end and the second end of the back plate are adjacent the first end and the second end of the body, respectively, such that the back plate extends generally parallel with the passageway. The back plate is formed of a substantially rigid material such that when a needle is inserted through the cannulation port and the self-sealing material the needle is inhibited or prevented from extending through the back plate.

An arteriovenous access graft is also provided and configured to be subcutaneously implanted in a subject between a first blood vessel and a second blood vessel of the subject such that blood flows through the graft from the first blood vessel to the second blood vessel. The arteriovenous graft comprises a flexible conduit having a first end and second end and defining a longitudinal flow passage between the first end and the second end. The first end is adapted to connect to an artery of the subject and the second end is adapted to connect to a vein of the subject such that blood flows through the flow passage of the conduit from the first end to the second end. A cannulation chamber comprises an elongated body having a first end and a second end and defining an annular passageway having a longitudinal axis extending between the first end and the second end. The body is configured to receive and surround a least a portion of the conduit in the passageway. The body comprises a flexible, nonporous elastomeric self-sealing material, and a cannulation port that exposes the self-sealing material. A flexible resilient elongated back plate having a first end and a second end is embedded in the body of the cannulation chamber with the first end and the second end of the back plate adjacent the first end and the second end, respectively, of the body such that the back plate extends generally parallel with the passageway. The back plate is formed of a substantially rigid material such that when a needle is inserted through the cannulation port and the self-sealing material the needle is inhibited or prevented from extending through the back plate.

In one embodiment, the body of the cannulation chamber includes an outer layer surrounding the cannulation chamber. The outer layer may comprise ePTFE.

In one aspect, the back plate may be planar. In another aspect, a transverse cross-section of the back plate is c-shaped, the back plate including a posterior wall and a pair of sidewalls extending from the posterior wall partially surrounding the passageway and defining an open anterior portion facing the cannulation port of the body.

In yet another aspect, the back plate comprises a plurality of independent identically shaped pieces being embedded in the body unconnected to and separate from adjacent pieces with spaces close enough between the pieces to prevent passage of a needle. Alternatively, the adjacent pieces partiality overlap one another.

In another embodiment, the plurality of pieces are connected at a midpoint by a longitudinal spine extending parallel with the back plate. Alternatively, the plurality of pieces are connected by a flexible material spanning the space between the adjacent pieces.

The back plate may have a plurality of openings small enough to prevent passage of a needle. The openings may be hexagonal.

In one embodiment, the back plate has opposed longitudinal side edges extending between the first end and the second end. Spaced linear blind slots extend orthogonally inwardly from the edges defining a zigzag pattern that zigzags transversely relative to the longitudinal axis between the side edges of the back plate.

The body of the cannulation chamber may be curved to have an arc angle formed by a longitudinal axis at the one end or another end of the curved chamber and an axis parallel to the longitudinal axis of a straight chamber that is between 10 and 30 degrees to accommodate placement in an extremity of the subject.

In a further aspect, the body has an outer surface including a continuous raised perimeter portion adjacent to the cannulation port such that the cannulation port can be tactilely or visually identified. Alternatively, the outer surface may include a pair of spaced parallel flanges adjacent to the cannulation port such that the cannulation port can be tactilely or visually identified and the cannulation chamber can be manipulated follow implantation.

There may be beading material disposed around a circumference of at least a portion of a length of the conduit.

### BRIEF DESCRIPTION OF THE DRAWINGS

For a more complete understanding of the vascular arteriovenous graft, reference should now be had to the embodiments shown in the accompanying drawings and described below. In the drawings:

FIG. 1 is a perspective view of an embodiment of a vascular arteriovenous graft.

FIG. 2 is a top plan view of the arteriovenous graft as shown in FIG. 1.

FIG. 3 is a side elevation view of the arteriovenous graft as shown in FIG. 1.

FIG. 4 is a longitudinal cross-section view of the arteriovenous graft as shown in FIG. 1 taken along line 4-4 of FIG. 3.

FIG. 5 is a transverse cross-section view of the arteriovenous graft as shown in FIG. 1 taken along line 5-5 of FIG. 2.

FIG. 6 is a top plan view of an embodiment of a flexible conduit for use in the arteriovenous graft as shown in FIG. 1.

FIG. 7 is a top perspective view of an embodiment of a flexible back plate for use in the arteriovenous graft as shown in FIG. 1.

FIG. 8 is a perspective view of an embodiment of a cannulation chamber for use with the arteriovenous graft as shown in FIG. 1 and shown in dashed lines except for the back plate as shown in FIG. 7 in solid lines.

FIG. 9 is another embodiment of an arteriovenous graft including two spaced cannulation chambers.

FIG. 10 is a close-up front perspective view of the cannulation chamber as shown in FIG. 8.

FIG. 11 is a perspective view of another embodiment of a flexible back plate for use in the arteriovenous graft as shown in FIG. 1.

FIG. 12 is a perspective view of a third embodiment of a flexible back plate for use in the arteriovenous graft as shown in FIG. 1.

FIG. 13 is a perspective view of a fourth embodiment of a flexible back plate for use in the arteriovenous graft as shown in FIG. 1.

FIG. 14 is a perspective view of a fifth embodiment of a flexible back plate for use in the arteriovenous graft as shown in FIG. 1.

FIG. 15 is a perspective view of a portion of a sixth embodiment of a flexible back plate for use in the arteriovenous graft as shown in FIG. 1.

FIG. 16 is a perspective view of a portion of a seventh embodiment of a flexible back plate for use in the arteriovenous graft as shown in FIG. 1.

FIGS. 17A-17C are top perspective, top plan and side elevation views, respectively, of another embodiment of a cannulation chamber for use with the arteriovenous graft shown in FIG. 1.

FIGS. 18A and 18B are a perspective view and a transverse cross-section view taken along line 18B-18B of FIG. 18A, respectively, showing a third embodiment of a cannulation chamber for use with the arteriovenous graft shown in FIG. 1.

FIGS. 19A and 19B are a perspective view and a transverse cross-section view taken along line 19B-19B of FIG. 19A, respectively, showing a fourth embodiment of a cannulation chamber for use with the arteriovenous graft shown in FIG. 1.

### DESCRIPTION

The present invention now will be described more fully with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art. In the drawings, like numbers refer to like elements throughout. Thicknesses and dimensions of some components may be exaggerated for clarity.

In addition, spatially relative terms, such as “under,” “below,” “lower,” “over,” “upper,” “downward,” “upward,” “inward, “outward” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

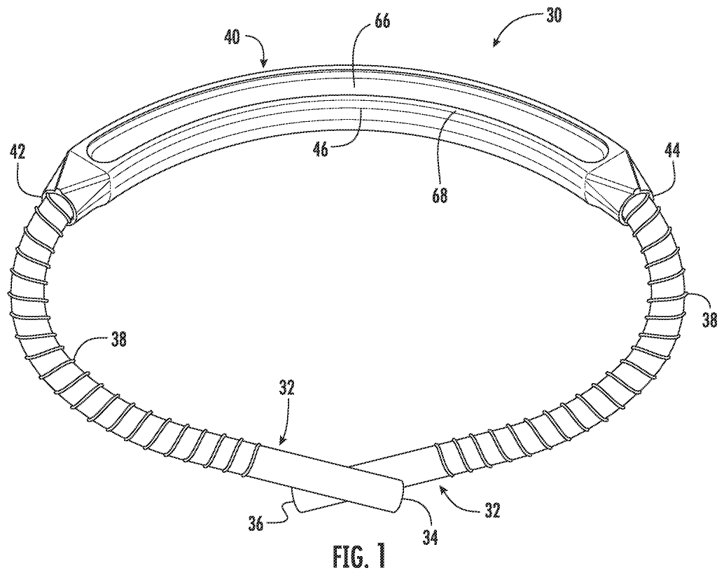

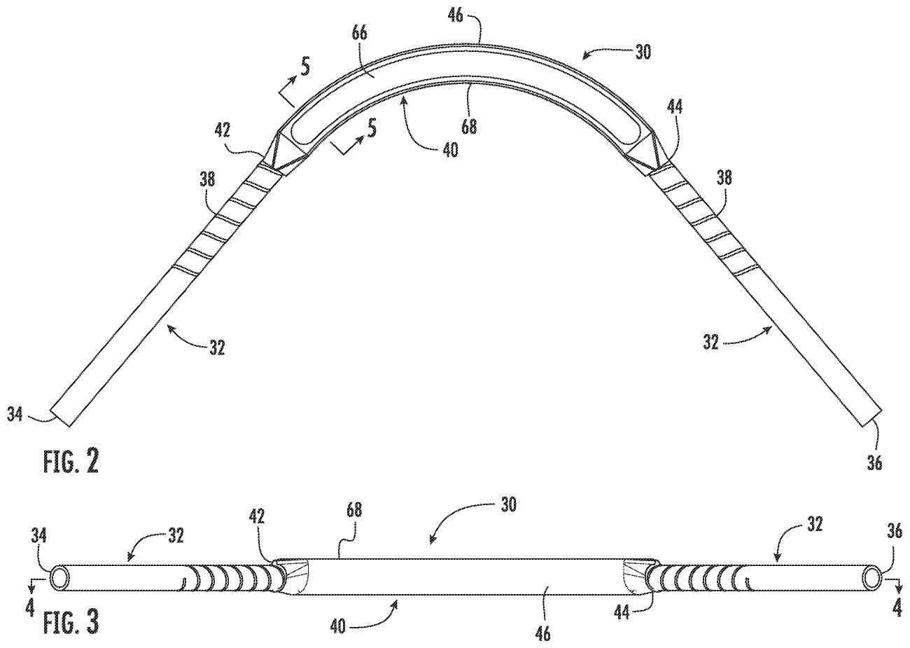

An embodiment of an arteriovenous graft is shown in FIG. 1 and generally designated at 30. The arteriovenous graft 30 is configured to be implanted in a subject. The AVG 30 comprises a conduit 32 having a first end portion 34 and a second end portion 36. The conduit 32 may be formed of an inert biocompatible material such as ePTFE, polyurethane, Dacron, or the like. The conduit 32 may also be formed of other biological materials, such as animal or human vessels, or biologically engineered tissue conduit. The first end portion 34 is configured to connect to a first blood vessel of a subject, such as an artery, at an end thereof. The second end portion 36 is configured to connect to a second blood vessel of the subject, such as a vein, at an end thereof. In this regard, blood flows through the conduit 32 from the first end portion 34 to the second end portion 36. The arteriovenous graft 30 could be used as an arterial-arterial graft when, for example, the vein could instead be an artery. Beading material 38 may be included on the outer periphery of the conduit 32. Such beading material may be in the form of PTFE wrapped around the outer surface in a spiral or helical configuration, which will provide some resistance to kinking or kink-resistant. One or both of the end portions 34, 36 of the conduit 32 may be corrugated. Other examples of external support or localized strain relief, especially at the intersection of the conduit 32 and the cannulation chamber 40, can be used, including rings, bushings at the Chamber body 46 to conduit 32 transition or other means to reduce kinking of the arteriovenous graft 30.

A cannulation chamber 40 is positioned between the first end portion 34 and the second end portion 36 of the conduit 32. The chamber 40 includes an inlet end 42 and an outlet end 44. The conduit 32 extends through the chamber 40 from the inlet end 42 to the outlet end 44. The chamber 40 comprises an elongated chamber body 46 that surrounds the conduit 32. The chamber body 46 defines the chamber inlet 42 and the chamber outlet 44. The chamber 40 further includes a flexible elongated back plate 50 embedded in the chamber body 46.

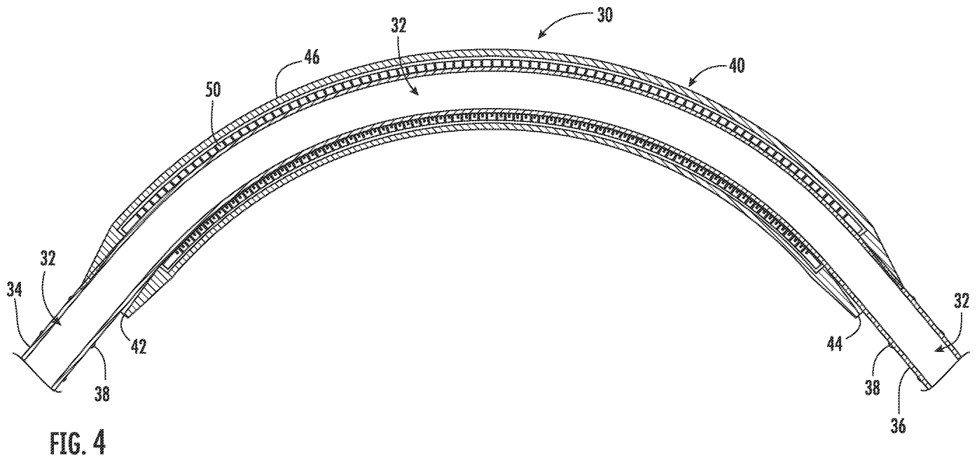

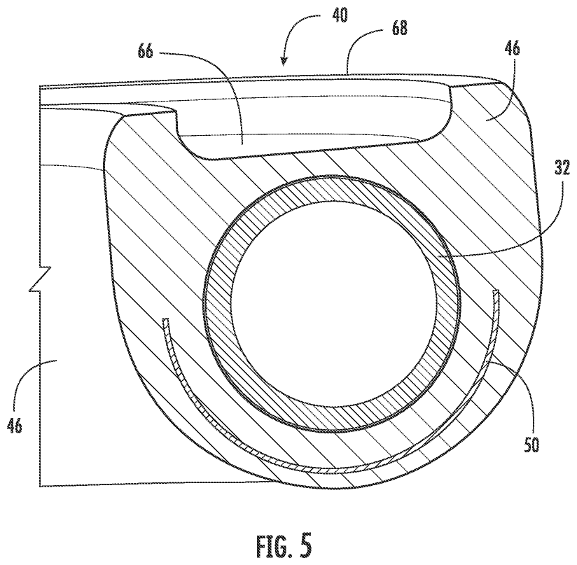

Cross-sectional views of the cannulation chamber 40 are shown in FIGS. 4 and 5. As illustrated, the chamber body 46 has inner surface 52 and an outer surface 54. An outer of layer of material, such as ePTFE, may be added to the chamber 40 to encourage tissue ingrowth and minimize foreign body reaction adjacent to the chamber. The inner surface 52 may define an annular fluid flow passageway having a longitudinal axis coaxial with a longitudinal axis of the chamber 40. The longitudinal passageway extends from the inlet end 42 to the outlet end 44 of the chamber body 46. The longitudinal passageway defines a longitudinal fluid flow path wherein blood may flow through. The longitudinal passageway has a circular or substantially circular cross-section. This configuration accommodates the conduit 32 having a similarly shaped flow path to minimize disturbance of laminar flow. The conduit 32 is shown in the drawings to be extending through the chamber body 46. The configuration can allow the conduit 32 to retain the circular or substantially circular cross-section or shape to inhibit flow disturbances therethrough.

The back plate 50 may be disposed between the inner surface 52 and the outer surface 54 of the cannulation chamber 40. Specifically, the chamber body 46 may be molded around the back plate 50. Alternatively, the back plate 50 may be adhered or otherwise attached to the inner surface 52. In turn, the inner surface 52 may be adhered or otherwise attached to the conduit 32. The chamber body 46 is formed of a flexible self-sealing material such as, but not limited to, silicone, which is a stretchable material that is suitable for repeated punctures. When the needle N is inserted through the self-sealing material, the self-sealing material is then able to self-seal after removal of the needle N. In various embodiments the self-sealing material 80 may have a thickness of between about 0.5 mm and about 10 mm and between about 1 mm and about 5 mm.

In some embodiments, the arteriovenous graft 30 may have a total extended length of between about 30 cm and about 80 cm. The end portions 34, 36 of the conduit 32 may each have a length of between about 5 cm and about 15 cm. The ends of the conduits may be trimmed or shaped in order to fashion an anastomosis. The ends of the conduits may also have a hooded configuration to present additional options for anastomosis creation.

The back plate 50 is formed of a substantially rigid biocompatible material such as, for example, biocompatible metals, including nitinol and titanium, or a substantially rigid polymer or composite, including thermoplastic polyurethane, silicone. Mesh or woven materials may also be used, such as Kevlar, chain mail, or other puncture resistant fabric. Rigid biological materials, such as connective tissue, are also possible. When a dialysis needle is inserted through the cannulation port 66 of the chamber 40, the needle is prevented or substantially prevented by the back plate 50 from extending through the posterior wall 60 or one of the side walls 62 of the chamber body 46. The back plate 50 may be of any shape, such as flat (FIG. 13), C-shaped or U-shaped in the manner of an open-ended semi-cylinder, so as to prevent the needle N from penetrating the back plate. With this C-shaped or U-shaped configuration, the back plate 50 includes a posterior wall 60 and opposing sidewalls 62 defining a cavity 64. The conduit 32 may be received in the cavity or recess 64 defined by the back plate 50. The back plate 50 thus surrounds about 180 degrees of the circumference of the chamber body 46 and the conduit 32. The back plate 50 has a length that is at least a substantial portion of the length of the chamber 40. In some embodiments, the length of the back plate 50 and the chamber 40 are substantially the same. The back plate 50, the conduit 32 and the chamber body 46 may be provided as an integrated cannulation chamber 40. The cannulation chamber 40 may be molded and then fit onto a graft conduit 32.

The cannulation chamber 40 has an open anterior portion including an aperture defining a cannulation port 66 configured to receive a dialysis needle there through. As described above, in some embodiments the outer surface 54 can include an additional layer of material, such as ePTFE or self-sealing material across the cannulation port 66. The chamber body 46 may include a raised perimeter or rim 68 defining the port 66 such that the cannulation port can be tactilely or visually identified when the AVG is implanted in a subject. That is, the raised perimeter may be visible through the skin of the subject or felt through the skin by medical personnel as a port locating feature.

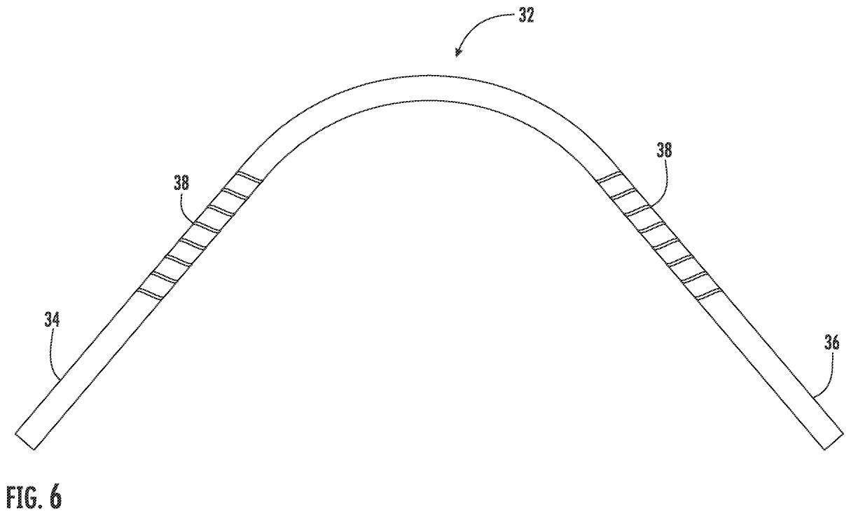

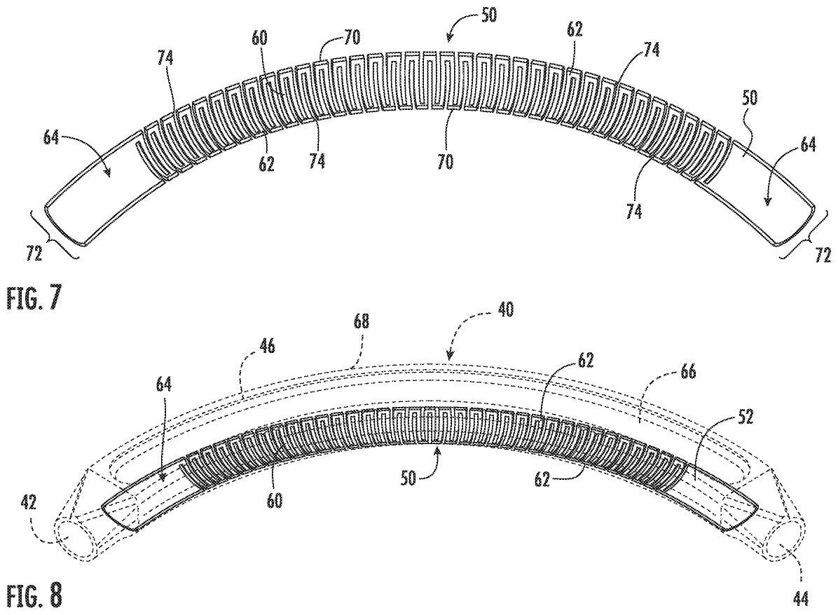

When a dialysis needle is inserted through the cannulation port 66, the needle may be inhibited or prevented from extending through the posterior 60 or the side walls 62 of the back plate 50. Referring to the back plate 50 as shown in FIGS. 7 and 8, the back plate 50 is an elongated flexible, resilient member configured to provide structural support to the cannulation chamber 40 while preventing needles from passing through the body. The back plate 50 comprises two opposed major longitudinal edges 70 substantially equidistant from one another along the length of the back plate. The end edges 72 of the back plate 50 are much shorter and extend between and interconnect the longitudinal edges 70 of the back plate 50. The back plate 50 has blind linear slots 74 extending orthogonally from the longitudinal edges 70 of the back plate 50. The slots 74 extend alternately from one edge and then from the opposite edge. The back plate 50 is curved such that the slots 74 beginning at the outer curved edge 70 of the back plate are about 2.7 mm wide. The slots 74 beginning at the inner edge of the curve of the back late 50 are about 0.8 mm wide. The slots 74 are thus small enough to prevent needle penetration, but still give the back plate 50 great flexibility.

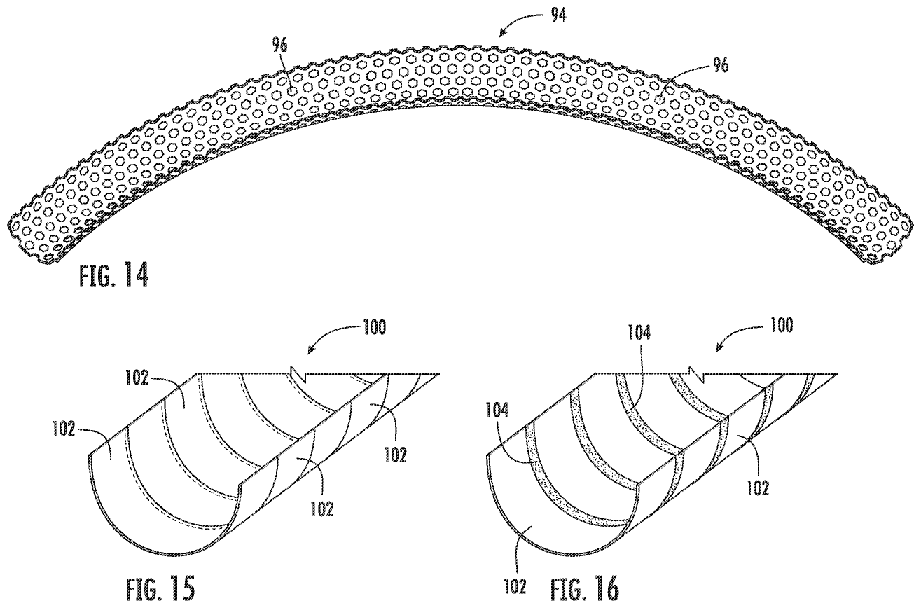

Another embodiment of a flexible, resilient back plate is shown in FIG. 11 and generally designated at 80. The back plate 80 comprises a plurality of identical u-shaped pieces 82 joined at their midpoint by a spine 84 running the length of the back plate. Other than connection at the spine 84, the pieces 82 are free from any other connection allowing for freedom of movement each of the pieces and for the back plate 80. Other embodiments of the back plate may be configured as a solid, C-shaped of U-shaped back plate 90 (FIG. 12) or substantially planar 92 (FIG. 13). In yet another embodiment, the concave solid back plate 94 (FIG. 14) may be perforated with a plurality of openings 96. The openings 96 of the back plate 94 are small enough to prevent needle breakthrough. In the embodiment shown in the figures, the openings 96 are hexagonal, such that the plurality of opening 66 along the back plate 94 resembles a honeycomb.

Referring now to FIG. 15, a still further embodiment of a back plate is shown and generally designated at 100. In this embodiment, the back plate 100 comprises individual c-shaped pieces 102 which, when molded into the cannulation chamber 40, overlap one another but are not directly connected. This arrangement provides freedom of movement to each piece 102 and the desired overall flexibility to the cannulation chamber 40. FIG. 16 shows a similar configuration, except that the individual pieces 102 are joined by a flexible material 104 that does not otherwise limit the relative movement of the pieces 102 and flexibility of the chamber 40.

As described hereinabove and shown in the drawings, the cannulation chamber 40 and associated back plates 50 may be curved to varying degrees to accommodate implantation in various locations throughout the body. The cannulation chamber is shown with the longitudinal passageway extending from the inlet end 42 through the outlet end 44. A curve angle, or an arc angle, is defined by the angle between the passageway extending from the inlet or outlet 42, 44 and an axis parallel to a longitudinal axis that would be defined by a “straight” cannulation chamber 40. The cannulation chamber 40 may generally be symmetrical; that is, the arc angles at each end may be equal. For the purposes of the present application, a chamber generally referred to as having an “arc angle” or a “curve angle” or being “curved” to a certain value (e.g., number of degrees) is a chamber that has equal or substantially equal angles A1 and A2. The chamber may be initially curved from between about 0 degrees and about 60 degrees. In other words, each of the arc or curve angles A1 and A2 may be between about 0 degrees and about 60 degrees. The curved chamber creates a curved longitudinal passageway or flow path through the chamber. The curved chambers may be configured such that surface area of the cannulation port 30 on the anterior outer surface of the housing provides an advantageously large “target” cannulation area, for example, between about 10 and about 30 degrees so as to be configured to be implanted in the arm of a subject. It is understood that because the cannulation chamber 40 is flexible that the curve angles may be changed or customized during implantation to accommodate the anatomical location and position of the graft 30. Moreover, the cannulation chamber 40 as described herein is flexible enough that the ends 42, 44 may meet such that the cannulation chamber 40 forms a closed loop. While this arrangement may not be necessary in application, it demonstrates the degree of flexibility of the cannulation chamber 40.

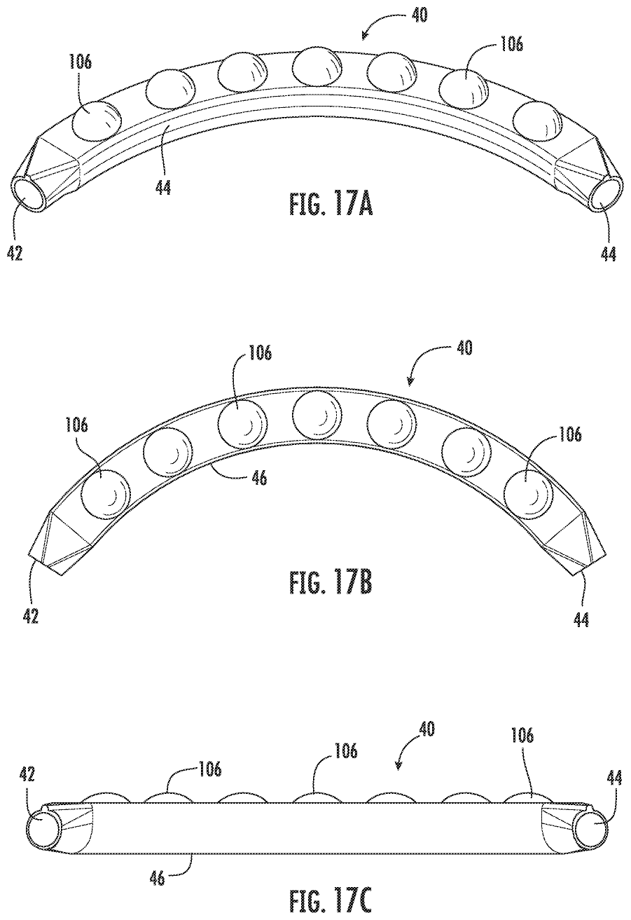

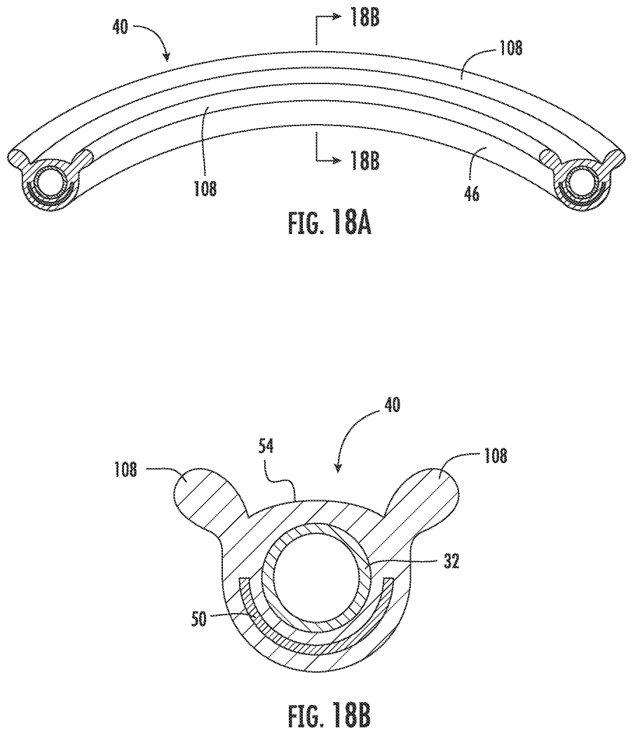

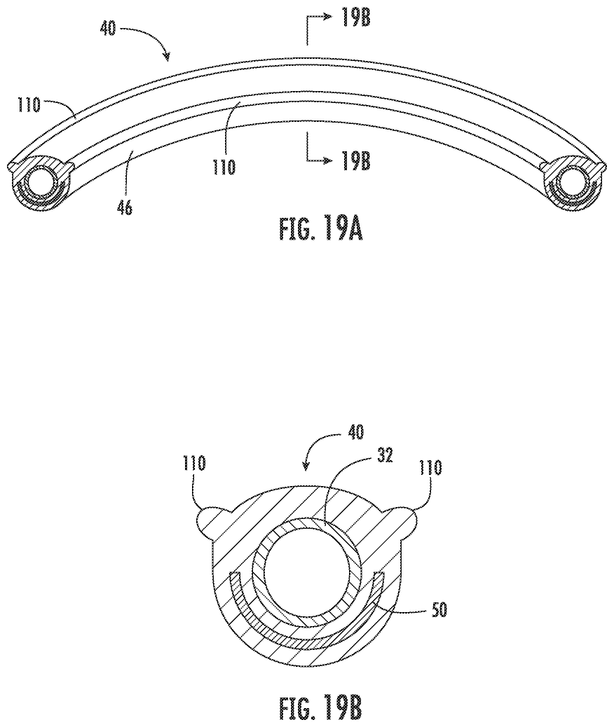

Referring to FIGS. 17A-17C, another embodiment of the cannulation chamber 40 is shown and comprises a plurality of longitudinally spaced domes along the anterior portion of the chamber body 46. The domes 106 replace the cannulation port 66 and provide tactile feedback to the user in determining a cannulation target. FIGS. 18A and 18B show a third embodiment of the cannulation chamber 40 including circumferentially spaced ears 108 replacing the cannulation port 66. The ears 108 allow handling of the cannulation chamber 40 for implantation and across the skin boundary when in a subcutaneous position to aid in cannulation. Also, in FIGS. 19A and 19 B, the ears 108 are replaced by rails 110 which are smaller and are circumferentially spaced further apart than the ears 108. The rails 110 along tactile feedback through the skin providing information of the rotational position of the cannulation chamber 40.

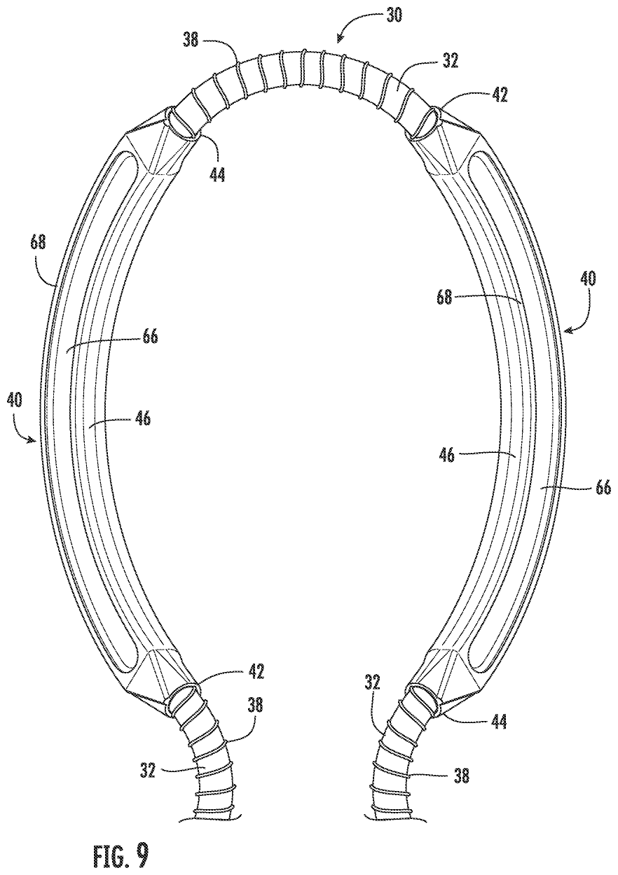

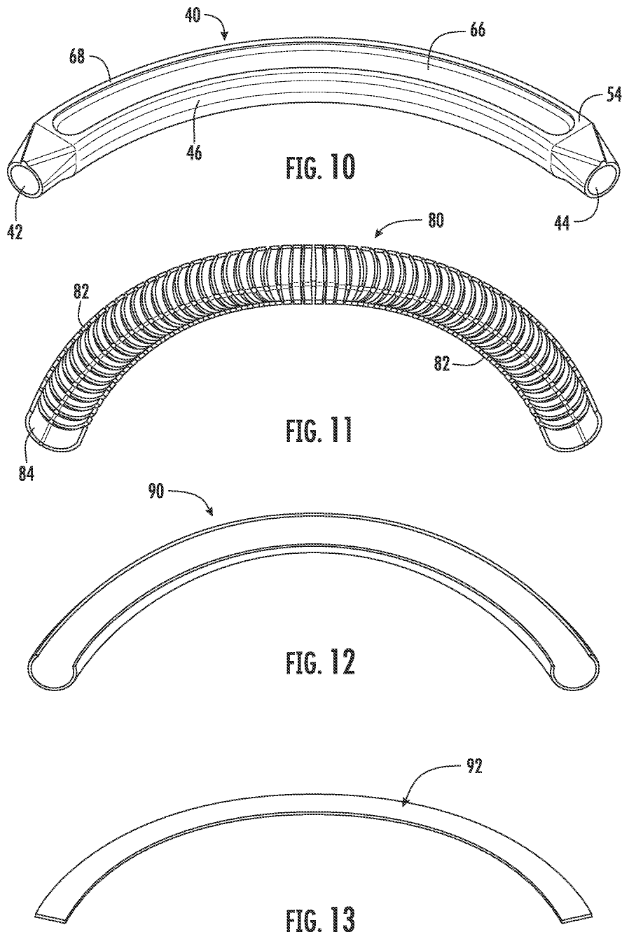

It is contemplated that the cannulation chamber 40 as described herein may be prepared separately from the conduit 32. It is also contemplated that various components described above may be supplied as a medical kit. For example, the chamber may be supplied with a conduit for later assembly and use. Each arteriovenous graft 30 may comprise two or more cannulation chambers (FIG. 9). The chambers may be identical or substantially identical. An intermediate portion of the conduit 32 is typically disposed between the chambers.

The arteriovenous graft 30 as described herein has many advantages, including a self-sealing, immediately usable graft that is flexible when the graft is implanted and maintained in a subject. The AVG may be bent or otherwise manipulated to accommodate a particular implantation site or geometry as the patient moves in daily life. The graft is versatile so as to be implanted in different or particular configurations in the body of a subject depending on the implantation location chosen based on suitable vascular anatomy. The arteriovenous graft owes its flexibility to the back plate which allows the cannulation chamber to flex while still resisting back or side wall needle puncture. Because the artervenous graft is flexible, the cannulation chamber can be larger since it conforms to the underlying patient anatomy. This allows for a longer and larger cannulation zone to help facilitate more frequent cannulation, for example, during Home Hemodialysis. Other embodiments as described herein create an enhanced tactile interface for even greater ease of finding where to cannulate the graft. Moreover, the embodiments of the artervenous graft are compatible with any ePTFE graft, biologic grafts, and fistulas. The arteriovenous graft may help prevent traumatic cannulations or graft degeneration so as to lead to higher patency rates for arteriovenous grafts, decrease the risk of hemorrhage or infection for hemodialysis patients, and reduce overall vascular access related healthcare costs.

We claim:

1. A cannulation chamber for use with an arteriovenous graft including a flexible conduit, the cannulation chamber comprising:
an elongated body having a first end and a second end and defining an annular passageway having a longitudinal axis extending between the first end and the second end, the body adapted to receive and surround a least a portion of the conduit in the passageway, the body comprising
a flexible, nonporous elastomeric self-sealing material, and
a cannulation port that exposes the self-sealing material; and
a flexible resilient elongated back plate having a first end and a second end, the back plate embedded in the body of the cannulation chamber with the first end and the second end of the back plate adjacent the first end and the second end of the body, respectively, such that the back plate extends generally parallel with the passageway,
wherein the back plate is formed of a substantially rigid material such that when a needle is inserted through the cannulation port and the self-sealing material the needle is inhibited or prevented from extending through the back plate.

2. The cannulation chamber as recited in claim 1, wherein the body includes an outer layer surrounding the cannulation chamber.

3. The cannulation chamber as recited in claim 2, wherein the outer layer comprises ePTFE.

4. The cannulation chamber as recited in claim 1, wherein the back plate is planar.

5. The cannulation chamber as recited in claim 1, wherein a transverse cross-section of the back plate is c-shaped, the back plate including a posterior wall and a pair of sidewalls extending from the posterior wall partially surrounding the passageway and defining an open anterior portion facing the cannulation port of the body.

6. The cannulation chamber as recited in claim 1, wherein the back plate comprises a plurality of independent identically shaped pieces being embedded in the body unconnected to and separate from adjacent pieces with spaces dose enough between the pieces to prevent passage of a needle.

7. The cannulation chamber as recited in claim 1, wherein the back plate comprises a plurality of independent identically shaped pieces being embedded in the body unconnected to adjacent pieces such that the adjacent pieces partially overlap one another.

8. The cannulation chamber as recited in claim 6, wherein the plurality of pieces are connected at a midpoint by a longitudinal spine extending parallel with the back plate.

9. The cannulation chamber as recited in claim 6, wherein the plurality of pieces are connected by a flexible material spanning the space between the pieces.

10. The cannulation chamber as recited in claim 5, wherein the back plate has a plurality of openings small enough to prevent passage of a needle.

11. The cannulation chamber as recited in claim 10, wherein the openings are hexagonal.

12. The cannulation chamber as recited in claim 5, wherein the back plate has opposed longitudinal side edges extending between the first end and the second end and spaced linear blind slots extending orthogonally inwardly from the edges defining a zigzag pattern that zigzags transversely relative to the longitudinal axis between the side edges of the back plate.

13. The cannulation chamber as recited in claim 1, wherein the body is curved to have an arc angle formed by a longitudinal axis at the one end or another end of the curved chamber and an axis parallel to the longitudinal axis of a straight chamber that is between 10 and 30 degrees to accommodate placement in an extremity of the subject.

14. The cannulation chamber as recited in claim 1, wherein the body has an outer surface including a continuous raised perimeter portion adjacent to the cannulation port such that the cannulation port can be tactilely or visually identified.

15. The arteriovenous graft as recited in claim 1, wherein the chamber body has an outer surface including a pair of spaced parallel flanges adjacent to the cannulation port such that the cannulation port can be tactilely or visually identified and the cannulation chamber can be manipulated follow implantation.

16. An arteriovenous access graft configured to be subcutaneously implanted in a subject between a first blood vessel and a second blood vessel of the subject such that blood flows through the graft from the first blood vessel to the second blood vessel, the arteriovenous graft comprising:
a flexible conduit having a first end and second end and defining a longitudinal flow passage between the first end and the second end, wherein the first end is adapted to connect to an artery of the subject and the second end is adapted to connect to a vein of the subject such that blood flows through the flow passage of the conduit from the first end to the second end;
a cannulation chamber comprising
an elongated body having a first end and a second end and defining an annular passageway having a longitudinal axis extending between the first end and the second end, the body configured to receive and surround a least a portion of the conduit in the passageway, the body comprising
a flexible, nonporous elastomeric self-sealing material, and
a cannulation port that exposes the self-sealing material; and
a flexible resilient elongated back plate having a first end and a second end, the back plate embedded in the body of the cannulation chamber with the first end and the second end of the back plate adjacent the first end and the second end, respectively, of the body such that the back plate extends generally parallel with the passageway,
wherein the back plate is formed of a substantially rigid material such that when a needle is inserted through the cannulation port and the self-sealing material the needle is inhibited or prevented from extending through the back plate.

17. The arteriovenous access graft of claim 16, further comprising beading material disposed around a circumference of at least a portion of a length of the conduit.

18. The arteriovenous access graft as recited in claim 16, wherein the body includes an outer layer surrounding the cannulation chamber.

19. The arteriovenous access graft as recited in claim 18, wherein the outer layer comprises ePTFE.

20. The arteriovenous access graft as recited in claim 16, wherein the back plate is planar.

21. The arteriovenous access graft as recited in claim 16, wherein a transverse cross-section of the back plate is c-shaped, the back plate including a posterior wall and a pair of sidewalls extending from the posterior wall partially surrounding the passageway and defining an open anterior portion facing the cannulation port of the body.

22. The arteriovenous access graft as recited in claim 16, wherein the back plate comprises a plurality of independent identically shaped pieces being embedded in the body unconnected to and separate from adjacent pieces with spaces dose enough between the pieces to prevent passage of a needle.

23. The arteriovenous access graft as recited in claim 16, wherein the back plate comprises a plurality of independent identically shaped pieces being embedded in the body unconnected to adjacent pieces such that the adjacent pieces partially overlap one another.

24. The arteriovenous access graft as recited in claim 22, wherein the plurality of pieces are connected at a midpoint by a longitudinal spine extending parallel with the back plate.

25. The arteriovenous access graft as recited in claim 22, wherein the plurality of pieces are connected by a flexible material spanning the space between the pieces.

26. The arteriovenous access graft as recited in claim 21, wherein the back plate has a plurality of openings small enough to prevent passage of a needle.

27. The arteriovenous access graft as recited in claim 26, wherein the openings are hexagonal.

28. The cannulation chamber as recited in claim 21, wherein the back plate has opposed longitudinal side edges extending between the first end and the second end and spaced linear blind slots extending orthogonally inwardly from the edges defining a zigzag pattern that zigzags transversely relative to the longitudinal axis between the side edges of the back plate.

29. The cannulation chamber as recited in claim 16, wherein the body is curved to have an arc angle formed by a longitudinal axis at the one end or another end of the curved chamber and an axis parallel to the longitudinal axis of a straight chamber that is between 10 and 30 degrees to accommodate placement in an extremity of the subject.

30. The cannulation chamber as recited in claim 16, wherein the body has an outer surface including a continuous raised perimeter portion adjacent to the cannulation port such that the cannulation port can be tactilely or visually identified.

31. The arteriovenous graft as recited in claim 16, wherein the chamber body has an outer surface including a pair of spaced parallel flanges adjacent to the cannulation port such that the cannulation port can be tactilely or visually identified and the cannulation chamber can be manipulated follow implantation.
